(19) United States
(12) Patent Application Publication
(10) Pub. No.: US 2025/0255942 A1
(43) Pub. Date: Aug. 14, 2025

(54) Clitoral Injection of Botulinum Toxin to Treat Sexual Dysfunction

(71) Applicant: Charles E. Runels, JR., Fairhope, AL (US)

(72) Inventor: Charles E. Runels, JR., Fairhope, AL (US)

(21) Appl. No.: 19/047,856

(22) Filed: Feb. 7, 2025

Related U.S. Application Data
(60) Provisional application No. 63/551,599, filed on Feb. 9, 2024.
(60) Provisional application No. 63/558,183, filed on Feb. 27, 2024.

(51) Int. Cl. A61K 38/48; A61K 9/00; A61K 35/19; A61P 15/00

(52) U.S. Cl. CPC A61K 38/4893; A61K 9/0019; A61K 9/0034; A61K 35/19; A61P 15/00; C12Y 304/24069

(57) ABSTRACT

Injecting botulinum toxin directly into the clitoris can treat various sexual dysfunctions and improve sexual satisfaction in many women. Without wishing to be bound by this hypothesis, these effects of botulinum toxin may act by, inter alia, increasing arterial blood flow within the clitoris via vasodilation, neovascularization within the clitoris, neurogenesis within the clitoris, changing autonomic tone, and activating the mid-hypothalamus.

The benefit of the 9 Feb. 2024 filing date of U.S. provisional patent application Ser. No. 63/551,599 is claimed under 35 U.S.C. § 119 (e). The benefit of the 27 Feb. 2024 filing date of U.S. provisional patent application Ser. No. 63/558,183 is claimed under 35 U.S.C. § 119 (e). The complete disclosures of both priority applications are hereby incorporated by reference in their entirety.

### TECHNICAL FIELD

This invention pertains to treating sexual dysfunction in a woman.

### BACKGROUND ART

Sexual dysfunction in both men and women can be a serious problem that can worsen family relations and psychological well-being. Moreover, sexual dysfunction in women is one of the only physical problems that is typically not diagnosed in the absence of accompanying secondary psychological distress.

As an example for comparison purposes, consider that men with erectile dysfunction or women with hypertension can be diagnosed with those disorders even if the patient experiences no psychological distress. By contrast, a woman who is not psychologically distressed will not meet currently-accepted criteria for sexual dysfunction even though she may suffer symptoms of dyspareunia, anorgasmia, decreased arousal, or decreased satisfaction. For example, women neither wanting to start a family nor desiring a sexual relationship may not be “distressed” and so are not counted as having sexual dysfunction even if they otherwise suffer all diagnostic symptoms.

This unique limitation in the definition of female sexual dysfunction partly accounts for the fact that the reported prevalence of sexual dysfunction is greater in older men and in younger women. To be blunt, older women who do not complain are simply not counted.

Notwithstanding this restriction in the currently-accepted definition of sexual dysfunction in women, in some reports, sexual dysfunction has been found in up to 40% of women.

There are currently limited treatment options for women. There is a disparity between not only in diagnostic criteria for sexual dysfunction in men and women but also in available treatment options. Consider the history of available treatments for men: Up through the 1980s, the prevailing wisdom was that 85% of men suffering from erectile dysfunction (ED) had psychogenic etiologies. Accordingly, urologists were encouraged to become skillful in sexual counseling. Then, following the discovery of the effects of phosphodiesterase-5 inhibitors (PDE5is), most cases of ED in men were found to have a neurovascular cause—a physical etiology rather than a psychological one. This new understanding that sexual dysfunction in men most often originated in the genitalia and not the brain triggered a proliferation of FDA-approved drugs and devices shown to improve sex for men. There are now over twenty FDA-approved drugs and devices in the United States for treating sexual dysfunction in men.

By contrast, there are currently only two FDA-approved oral medications with on-label indications to improve sex for women: flibanserin and bremelanotide. Both drugs are approved only for the treatment of hypoactive sexual desire disorder in premenopausal women. Both have a psychoactive mechanism of action with no direct effect on the female genitalia.

Even in the endocrine arena, there is only one FDA-approved drug for sexual dysfunction-prasterone, which is approved only for dyspareunia. There is no FDA-approved form of testosterone for women; this medication, which has been approved for men, is used off-label to treat millions of women who have benefited from this proven therapy for female sexual dysfunction.

Topical lubricants and anesthetics can be used to treat dyspareunia and deficits in arousal. However, these options basically treat symptoms, not the underlying etiologies. In limited cases, surgical therapies can help with some types of dyspareunia: surgery to treat phimosis of the clitoral hood can improve sensation; colporrhaphy and other surgeries can improve sensation; and labiaplasty can improve self-image. However, these surgeries are relatively uncommon, and most women suffering from sexual dysfunction have etiologies that lack any curative surgery.

Studies have shown that properly-injected platelet-rich plasma (PRP) can improve sexual function in women. Injection of platelet-rich plasma can cause neurogenesis and neovascularization in the clitoris, and thereby to improve sexual function. PRP, however, is an autologous blood isolate; it is not a drug, and the FDA does not regulate its use.

There are major disparities in currently-available therapies and medications for helping women and men with sexual dysfunction.

The discrepancy in available options in FDA-approved drugs for treating sexual dysfunction in men and women has resulted, at least in part, from significant differences in the approval criteria: A new drug for men need only improve erection firmness or the angle of deformity, which are physical and objective measurements. However, the FDA requires that a new drug for women must improve satisfaction, a psychological and subjective measurement. This subjective criterion is more difficult to reliably measure and demonstrate.

This difference in the approval criteria disincentivizes pharmaceutical companies to invest the money needed for research to bring new drugs to market to help women with sexual dysfunction.

Following is a partial list of FDA-approved, on-label drugs to help men with sexual dysfunction: PDE5is include Sildenafil (Viagra), Vardenafil (Levitra, Staxyn), Tadalafil (Cialis), Avanafil (Stendra). In the testosterone arena, there are Androderm (transdermal system), AndroGel (transdermal gel), Aveed (intramuscular injection), Depo-Testosterone (intramuscular injection), Fortesta (transdermal gel), Jatenzo (oral capsules), Kyzatrex (oral capsules), Natesto (nasal gel), Testim (transdermal gel), Testopel (pellet for subcutaneous implantation), Tlando (oral capsules), Vogelxo (transdermal gel), and Xyosted (subcutaneous injection autoinjector). Men also have a variety of vacuum erection devices, constriction devices, and traction devices. Also available are drugs for intracavernosal injection, including Viberect and Caverject. In addition, Muse and Edex are two vasoactive drugs administered as urethral suppositories. Xyflex and other drugs are approved for Peyronie's. And finally, there are a variety of FDA-approved devices available for penile implant. The preceding is only a partial list of approved, on-label treatments available for treating sexual dysfunction in men.

That men currently have 20-plus drugs and devices that directly improve their sexual lives could fail to suggest there is a disparity between the sexes in this regard, only by assuming that the vagina acts merely as a passive receptacle, and that no components of the female genitalia are ever involved in sexual dysfunction. Such a conclusion not only defies common sense, but it would also ignore the high degree of commonality in the embryology, histology, and anatomy of the male and female genitalia, the involvement of the autonomic nervous system in both, and their similar innervations and vascularizations.

The clitoris includes an external short glans attached to the long body of the corpus cavernosa, which is internally located and bifurcates along the pubic rami. The body and corpus cavernosa of the clitoris are made of erectile tissue with vascular spaces that can admit blood from arterioles during engorgement. The clitoris is richly innervated with sensory nerves. The major nerve that transmits sensations is a branch of the pudendal nerve known as the dorsal nerve of the clitoris. The cavernous nerves also traverse the clitoris and communicate with the autonomic nervous system.

Clitoral erection (or tumescence) results from a complex interaction of physiological, psychological, neural, vascular, and endocrine factors and is usually, though not exclusively, associated with sexual arousal. Clitoral erection occurs when an increase in blood flow from the dorsal clitoral and cavernosal clitoral arteries fills sinusoids contained in two expandable erectile structures called the corpora cavernosa. The increase in clitoral blood supply is a result of arterial vasodilatation and smooth muscle relaxation in the corpus cavernosum. This blood engorgement leads to increased clitoral size and tumescence. Clitoral tumescence results in extrusion of the glans clitoris, and thinning of the skin enhances sensitivity to physical stimulation. Physical stimulation of the tumescent clitoris provokes pelvic floor muscle contraction and triggers orgasm. After orgasm, the clitoral erection usually ends (detumescence), but this may take time. Clitoral tumescence is also associated with increased blood supply to the vulva, vaginal transudation (secretion of moisture through the vaginal walls, which serves as lubrication), nipple erection, and prolonged body relaxation. As such, failure to achieve clitoral tumescence may be an important factor in female sexual dysfunction.

There is an unfilled need for new and improved drug therapies for women that work by directly affecting the genitalia to improve sexual function, especially sexual satisfaction. Easier regulatory approval might be expected for a drug that: (1) has a known safety profile for women of childbearing age, and (2) has already been approved for other indications. Such a drug could potentially be repurposed and approved for a new indication—e.g., to improve arousal, orgasm, lubrication, and sexual satisfaction in women.

Botulinum toxin (BoNT) is well-known for its uses in muscle spasticity, strabismus, cosmetic applications, migraine treatments, and other uses. Because BoNT can block nerve conduction, in the sexual dysfunction arena it has previously been studied to help with problems that might be improved with less muscle contraction or with less sensation. For example, BoNT has been used to help with vaginismus and pelvic floor pain by relaxing muscles and by blocking nerve signaling at the motor end plate. Botulinum toxin has also been used to treat detrusor muscle instability by direct injection into the smooth muscle of the bladder wall for treating bladder spasms, overactive bladder, and neurogenic bladder dysfunction—by blocking nerve transmission, causing the relaxation of smooth muscle and relieving pain.

BoNT has also been studied as a potential means for lessening sensation, orgasm, and pain in the treatment of persistent genital arousal disorder (PGAD) and to help with vulvodynia. For both these indications, benefits, when demonstrated, have been attributed to BoNT's ability to interfere with nerve conduction.

Multiple studies since the 1950s have shown that botulinum neurotoxin promotes neurogenesis and neovascularization and, thereby, helps heal surgical, traumatic, and chronic wounds.

To summarize, prior uses of BoNT have been directed to lessening muscle contraction by direct injection of the muscle or to lessening sensation (including pain, arousal, and orgasm) by injection near the affected nerves. There are no previous known reports describing or suggesting the use of BoNT for directly increasing sensation, desire, arousal, orgasm, or sexual satisfaction in women.

There are no known reports of injecting BoNT into the clitoris—for any purpose. Previously, BoNT has been injected directly intramuscularly to relax muscles and treat vaginismus, pelvic floor pain, and bladder spasms. For persistent genital arousal disorder, BoNT has been injected near (but not into) the clitoris. To relieve vulvodynia, BoNT has been injected directly into the area of pain.

BoNT changes autonomic tone and increases arterial blood flow. BoNT has been injected into the corpus cavernosum in men to treat ED. The mechanism of action to improve ED is thought to be the relaxation of the smooth muscle of the arterioles, with an increase in arterial blood flow. Likewise, PDE5is also work by relaxing the smooth muscle controlling arterial blood flow into the penis, resulting in a firmer erection. So, at least in men, BoNT and PDE5is have been understood to have a similar mechanism of action. However, injection of BoNT into the corpus cavernosum of the clitoris has not been previously reported.

U.S. patent publication 2012/0251518 speculated, without presenting any supporting data, about certain potential uses of botulinum toxin for treating certain sexual dysfunctions.

U.S. Pat. No. 8,697,090 discloses the use of botulinum toxin to treat persistent genital arousal disorder.

C. Runels, “Botulinum Toxin for Female Sexual Dysfunction-A Review (ISCG 2023),” oshot.info/members/botox-for-female-sexual-dysfunction-a-review-iscg-2023/(March 2023) is a related presentation that was given by the present inventor.

R. Gari et al., Use of Botulinum Toxin (Botox®) in Cases of Refractory Pelvic Floor Muscle Dysfunction. Sex Med Rev. 2022 January; 10 (1): 155-161 (2021) pubmed.ncbi.nlm.nih.gov/34362710/discloses the use of Botulinum toxin A to treat female pelvic floor muscle dysfunction.

P. Pacik P T et al., Vaginismus Treatment: Clinical Trials Follow Up 241 Patients. Sex Med. 2017 June; 5(2):e114-e123. www.ncbi.nlm.nih.gov/pmc/articles/PMC5440634/discloses the use of Botulinum toxin to treat vaginismus. See also B. Jones, Vaginismus: Getting Vaginal Pain Validated and Seeking Treatment (webpage, downloaded 2024) www.verywellhealth.com/using-botox-to-treat-vaginismus-4139797; and A. Velayati et al., Can Botox Offer Help Women With Vaginismus? A Systematic Review and Meta-Analysis. International Journal of Sexual Health. 31. 233-243 (2019) www.researchgate.net/publication/333880074_Can_Botox_Offer_Help_Women_With_Vaginismus_A_Systematic_Review_and_Meta-Analysis.

J. Weinberger et al., Female Sexual Dysfunction: A Systematic Review of Outcomes Across Various Treatment Modalities, Sex. Med. Rev. 7(2), pp. 223-250 (2019) www.sciencedirect.com/science/article/abs/pii/S2050052118300015 presents a review of 103 published treatments for female sexual dysfunction, including 5 studies involving botulinum toxin A.

D. Morrissey et al., Botulinum Toxin A Injections Into Pelvic Floor Muscles Under Electromyographic Guidance for Women With Refractory High-Tone Pelvic Floor Dysfunction: A 6-Month Prospective Pilot Study. Female Pelvic Med Reconstr. Surg. 2015 September-October; 21 (5): 277-82. pubmed.ncbi.nlm.nih.gov/25900057/discloses the use of botulinum toxin to treat pelvic floor dysfunction.

M. Moga et al., Therapeutic Approaches of Botulinum Toxin in Gynecology. Toxins (Basel). 2018 Apr. 21; 10(4):169 www.ncbi.nlm.nih.gov/pmc/articles/PMC5923335/discloses the use of botulinum toxin isoforms (particularly isoform A) for treating gynecological pathologies, including myofascial pelvic pain, vaginism, dyspareunia, vulvodynia, and overactive bladder or urinary incontinence.

Application of BOTOX in Female Sexual and Genitourinary Dysfunction (webpage, downloaded 2024) www.excelmale.com/threads/application-of-botox-in-female-sexual-and-genitourinary-dysfunction.23530/and B. Dick et al., Application of Botulinum Neurotoxin in Female Sexual and Genitourinary Dysfunction: A Review of Current Practices. Sexual Medicine Reviews. 9. 10.1016 (2020) www.researchgate.net/publication/340270621_Application_of_Botulinum_Neurotoxin in_Female_Sexual_and_Genitourinary_Dysfunction_A_Review_of_Current_Practic es provide overviews of several studies using botulinum toxin to treat dyspareunia, vaginismus, vestibulodynia, and persistent genital arousal disorder.

C. Petersen et al., Botulinum Toxin Type A-A Novel Treatment for Provoked Vestibulodynia? Results from a Randomized, Placebo Controlled, Double Blinded Study, The Journal of Sexual Medicine, Volume 6, Issue 9, September 2009, Pages 2523-2537 academic.oup.com/jsm/article-abstract/6/9/2523/6834376?redirectedFrom=fulltext&login=false discloses that, as compared to placebo, injecting botulinum toxin in the vestibule of women diagnosed with vestibulodynia did not reduce pain, nor did it improve sexual functioning, nor did it impact quality of life.

### SUMMARY OF THE INVENTION

I have discovered that injecting botulinum toxin directly into the clitoris can increase sexual arousal, sensation, orgasm, and satisfaction in many women. Without wishing to be bound by this hypothesis, these effects of BoNT are believed to act by, inter alia, changing autonomic tone, increasing arterial blood flow, and possibly by neovascularization and neurogenesis.

Previously-reported work with BoNT has been based primarily on the premise that pain can be reduced by relaxing the affected muscles. Thus, it was surprising that BoNT would have any effect on the clitoris, which lacks any muscles. It was further surprising that a composition (BoNT) known for use in blocking motor muscles can improve nerve responsiveness in the clitoris.

BoNT has previously been used in some applications to reduce sensation, so it is especially surprising that BoNT can actually increase sensation in women when used in accordance with the present invention.

Without wishing to be bound by this hypothesis, it is believed that BoNT injected into the clitoris primarily acts centrally, by affecting the autonomic nervous system and the hypothalamus, and that these effects improve female sexual function. A possible additional or alternative mechanism is a local effect, in which BoNT increases arterial circulation and engorgement of the clitoris, which also leads to improved sexual function. BoNT may perhaps also trigger neovascularization and neurogenesis, which could also improve sexual response and function.

Preliminary data have confirmed that BoNT injected directly into the clitoris improves female libido and orgasm, as well as satisfaction. These findings represent a new breakthrough in the treatment of women's sexual function.

BoNT has a proven track record for safety in clinical practice (when used for other purposes). In a prototype study, women who presented with complaints of anorgasmia, difficulty with orgasm, or other symptoms related to sexual dysfunction were successfully treated with BoNT injections into the clitoris.

Without wishing to be bound by this hypothesis, I propose that botulinum neurotoxin increases parasympathetic tone in women, and acts through an alternative mechanism that is not afforded by PDE5is, by not only increasing blood flow to the clitoris but also by affecting libido through axonal transport of BoNT and the resultant effects on sympathetic nervous system with an attenuation of the sympathetic nervous system and a relative enhancement of the parasympathetic nervous system.

Moreover, the autonomic nerves of the pelvis connect to the pelvic ganglion, thereby affecting signals to the hypothalamus. The hypothalamus is largely responsible for activating the autonomic, non-voluntary, physiological, sexual responses of arousal, lubrication, and orgasm and for signaling for the cerebral perception of arousal and sexual pleasure.

The hypothalamus also connects to the pituitary gland and affects hormone production. Through its effects on the parasympathetic nervous system and the hypothalamus, BoNT could change not only immediate emotions but also the production of pituitary hormones. Such hormonal changes may not be confined to the hormones FSH, LH, and estradiol, but may also extend to dopamine and serotonin balance, which profoundly affect orgasmic function.

To the inventor's knowledge, injecting the clitoris with BoNT to increase blood flow (viz., by dilating arterioles), or to increase parasympathetic tone, or to stimulate the mid-hypothalamus has never been previously suggested. To the inventor's knowledge, injecting the clitoris with BoNT to treat sexual dysfunction in women has never been previously suggested.

In view of the history of using botulinum toxin either for decreasing nerve signals or to decrease sensation—it is counterintuitive to instead inject BoNT directly into the clitoris to treat sexual dysfunction. Even though BoNT has been widely available to gynecologists for some time (e.g., it has typically been kept in the office for the past two decades for treating bladder spasms), there are no prior published reports of injecting BoNT directly into the clitoris to increase sensation, arousal, orgasm, and satisfaction in women. Even though the FDA has approved drugs for injection directly into the penis, there are no previously-reported studies known to the inventor that teach or suggest injecting any drug whatsoever directly into the clitoris. (To clarify: Autologous platelet-rich plasma (PRP) has previously been injected into the clitoris, but PRP is not considered a drug.) In particular, there are no previous reports of injecting BoNT directly into the clitoris for any purpose.

It was, therefore, quite surprising to discover that injecting BoNT directly into the clitoris can actually improve symptoms for women suffering from sexual dysfunction.

Without wishing to be bound by this hypothesis, I propose that botulinum neurotoxin injected into the clitoris increases parasympathetic tone in women, and that it thus can act in a way not afforded by PDE5is, by not only increasing blood flow to the clitoris but also by affecting libido through the changes in the autonomic nervous system and secondary changes in the hypothalamus.

BoNT may also exert vasodilatory effects by regulating arterial smooth muscle contraction via the eNOS/sGC/cGMP pathway, which increases endothelial nitric oxide. This could also lead to increased tumescence in the cavernosal tissue of the female clitoris.

The present invention achieves results that are contrary to what would have previously been expected. All known previous uses of BoNT in the female genitalia were intended to attenuate sensation rather than enhance it, by either decreasing arousal (by decreasing sensation), by decreasing muscle tone (by decreasing the signal at the motor end plate), or by decreasing pain (by decreasing sensation or muscle tone). It was surprising to discover that, when properly administered, BoNT can instead have the opposite effect of enhancing sexual responsiveness and pleasure.

The invention may be used to benefit women who wish to see an improvement in in libido, arousal, orgasm, and satisfaction, particularly for women for whom hormonal strategies were not wanted or whose hormones were already at acceptable levels (and therefore did not need hormonal therapies). Some women choose not to risk the side effects that might be seen with flibanserin and bremelanotide, which are arguably more troubling than the side effects expected with BoNT at doses used for the treatment of sexual dysfunction in accordance with the present invention.

With aging, there is a decrease in the number of nerves in the clitoris and a decrease in blood flow to the clitoris. Trauma, even from riding a bicycle, can damage nerves, resulting in decreased sensation from the clitoris. Without wishing to be bound by this hypothesis, injecting the corpus cavernosum of the clitoris with BoNT may increase the number of nerves and the number of blood vessels, thus improving sensation and sexual function.

In a preliminary study, several women who presented with complaints of anorgasmia, difficulty with arousal, or other symptoms related to sexual dysfunction were offered BoNT injections into the clitoris, and their responses were recorded.

All women were informed that they could also choose to receive no treatment at all. Offered the option, some of the women chose to have the BoNT combined with autologous PRP (rather than with saline alone). The co-administration with PRP may produce a synergy that enhances the intended response.

### BRIEF DESCRIPTION OF THE DRAWINGS

FIG. 1 depicts overall FSFI scores for 13 women in a preliminary trial, both before and after receiving the BoNT treatment.

FIG. 2 depicts overall changes in FSFI in the preliminary trial, as a function of each patient's age.

FIG. 3 depicts overall changes in FSFI in the preliminary trial, for BoNT administered in saline alone, and for BoNT administered with PRP.

FIG. 4 compares the overall changes in FSFI scores found in the preliminary trial reported here using BoNT, with those reported from a metanalysis involving two other prior treatments, namely flibanserin and bremelanotide.

### MODES FOR CARRYING OUT THE INVENTION

### Abbreviations

BoNT: botulinum neurotoxin
NT: neurotoxin
FSFI: female sexual function index
PRP: platelet-rich plasma
ED: erectile dysfunction
PGAD: persistent genital arousal disorder
PD5i: phosphodiesterase-5 inhibitor

### Methods

A pilot study was conducted with women wishing to improve sexual function. Thirteen women, ages 32-64, presented with complaints of sexual dysfunction (desire, arousal, orgasm, or satisfaction). The patients were seen in a clinical private practice and were paid neither to participate in the procedure nor to complete the associated pre- and post-treatment surveys. All patients were fully informed of the innovative therapeutic, off-label, and experimental nature of the procedure, and all gave informed written consent for the procedure.

Exclusion criterion: all participants were without thrombocytopenia, myasthenia gravis, ongoing infection, pregnancy, inappropriate affect, high-dose corticosteroids, or clitoral phimosis.

Materials and equipment included the following: (1) 5-cc syringes, (2) 3-cc syringes, (3) 30-gauge, ½ inch, BD™ needles, (3) 18-gauge needles, (4) bacteriostatic saline, (5) Regen® PRP centrifuge tubes, (6) Regen® PRP centrifuge, (6) calcium chloride (CaCl₂)) 10% solution, (7) 30% lidocaine ointment, (8) Luer-Lok™ connectors, and (9) incobotulinumtoxinA (Xeomin®).

Meticulous attention should be paid both to the preparation of BoNT and to the method of injection to minimize pain and to enhance the likelihood of a positive outcome. When PRP is added, attention should be paid to the preparation of the PRP (including activation and anticoagulant).

### Anesthesia

First, the clitoral hood was retracted, and lidocaine ointment was applied to the body of the clitoris; some inadvertent application to the glans is inevitable, but the distal body of the clitoris should be covered. Injection was then delayed until 20 minutes after the application of the lidocaine ointment to allow for complete or near-complete anesthesia.

Note: Should the clitoral hood refuse retraction sufficient to visualize the body of the clitoris (only the glans is revealed), then the procedure should be aborted, and instead, the woman should be considered for surgical treatment for often-overlooked clitoral phimosis (which itself is a possible cause for sexual dysfunction).

### Preparation of the BoNT

Using an 18-gauge needle on a 1-cc syringe with a Luer-Lok™ connector, 1.0 cc of bacteriostatic saline was added to a 100-unit vial of BoNT (Xeomin®). Then 0.5 cc of the reconstituted BoNT (50 units) was drawn back into the syringe, and the needle recapped. The 50-unit syringe was then set aside until needed. (A 1-cc syringe was used since a larger syringe could result in less accurate measure of the solution.)

### Preparation of PRP

For each of the seven women who elected to combine BoNT with PRP, 10.0 cc of blood was drawn from the arm and centrifuged using a Regen® kit, producing around five cc of PRP supernatant. (The yield of PRP is hematocrit-dependent.) The Regen® tube was inverted five times (to resuspend platelets that might have adhered to the gel), and then 2.0 cc of PRP were drawn from the Regen® tube into a 3-cc syringe with an 18-gauge needle.

Next, a third syringe (1 cc with a Luer-Lok™ connected to an 18-gauge needle) was used to withdraw 0.1 cc of 10% CaCl₂) solution—and was set aside until needed.

Alternative Methods for Injecting the Clitoris—with or without PRP

Twenty minutes after application of the lidocaine ointment, the clitoris was injected with 50 units of BoNT, with or without PRP:

Injection of BoNT in Saline, without PRP

For the six women in the saline-only group (no PRP), 2.0 cc of saline were drawn into a new 3-cc syringe. Then 0.5 cc of BoNT solution (from step A) was attached using a Luer-Lok™ connector; the 0.5 cc of BoNT solution was transferred into the 3-cc syringe—resulting in 50 units of BoNT in 2.5 cc of saline (0.5+2.0) held by a 3-cc syringe.

Then, the Luer-Lok™ and the now-empty 1-cc syringe were replaced by a ½ inch, 30-gauge needle.

Then, the clitoral hood was retracted to expose the body of the clitoris, which was cleansed with gauze soaked with hypochlorous solution: Puracyn®: Hypochlorous Acid (HOCl) 0.012%, Electrolyzed Water (H₂0) 99.916%, Sodium Chloride (NaCl) 0.031%, Sodium Hypochlorite (NaOCl) 0.001%, Phosphates 0.040%.

A 30-gauge needle on the 3-cc syringe was inserted into the body of the clitoris at the 2 o'clock position, approximately 3 mm proximal to the glans. The 2.5 cc of saline with 50 units of BoNT were then injected. Only one side of the clitoris was injected. This step takes 15 to 30 seconds, with attention kept on the surrounding area. Swelling around the clitoris indicates injection has occurred into the surrounding area, rather than the clitoris itself. When performed properly, there should be no edema surrounding the clitoris, whether during or after injection.

During the injection, only the bevel of the needle was inserted into the tissue of the clitoris (no deeper), and the fluid was meticulously and slowly injected so that it was completely absorbed into the clitoris. Little to no edema was observed-neither in the area surrounding the clitoris nor in the clitoris itself, indicating that the material hydrodissected deeply along the corpus cavernosum, and not laterally into surrounding tissue.

Injection of BoNT Combined with PRP

For the seven women receiving BoNT combined with PRP, the 18-gauge needle was removed from the 3-cc syringe containing 2.0 cc of PRP (previously prepared), and a Luer-Lok™ connector was used to facilitate the addition of 50 units of BoNT (previously prepared).

Then, 0.1 cc of 10% CaCl₂) (previously prepared) was added to the mixture through the same connector, which was then replaced with a ½-inch 30-gauge needle.

Within 3 minutes of combining the three components (BoNT, PRP, and CaCl₂)), the woman's clitoris was injected with the mixture as otherwise described above for the women receiving injection without PRP.

Note: Waiting longer than 3 minutes after adding the CaCl₂) to PRP is not preferred, as the calcium can trigger the thrombin cascade, causing PRP to form a platelet-rich fibrin matrix (PRFM). If this should occur before the material has been injected, it will not pass through a 30-gauge needle, and the procedure should be aborted.

### Post-Injection Instructions

Each patient was encouraged to cleanse her clitoris and the surrounding area of the remaining lidocaine ointment upon arrival home, and then to proceed with usual daily activities, including sex. The patients were specifically told that there was no need for downtime, and that it was acceptable to resume sexual activity even the same day as the treatment. Patients receiving BoNT combined with PRP were also instructed to avoid non-steroidal anti-inflammatory drugs for one week.

### Ethics

This study fell in the category of Medical Practice and Innovative Therapy, an activity designed solely to benefit individual patient(s), which accordingly did not require IRB review (per the policies of the University of Virginia's Institutional Review Board for Health Sciences Research, which were followed here; the University of Virginia itself had no involvement in this study, however).

### Data Collection

The standardized Female Sexual Function Index (FSFI) questionnaire was used to measure arousal, desire, pain, orgasm, satisfaction, and lubrication. Outcomes measured the patients' sexual responses using self-administered FSFI surveys both before, and 4-12 weeks after treatment.

### Results

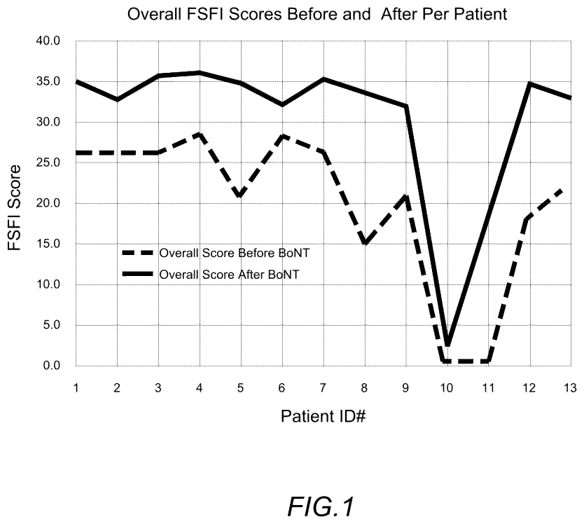

A total of 13 women, from 32 to 64 years old, with an average age of 50, presented to a private clinic seeking improved sexual function. Table 1 summarizes their responses to the FSFI questionnaire on the day of treatment, and 4 to 12 weeks later. An entry with an asterisk * indicates a patient who received PRP. The results of this study are also summarized in FIG. 1.

TABLE 1

| Patient | | | | | | | | Overall | Overall | Overall |
|---|---|---|---|---|---|---|---|---|---|---|
| ID | Domain | Desire | Arousal | Lubrication | Orgasm | Satisfaction | Pain | Pre-tx | Post-tx | Delta |
| 1 | Pre-tx | 4.8 | 4.2 | 3.9 | 4.8 | 4 | 4.8 | 26.5 | | |
| | Post-tx | 6 | 6 | 4.8 | 6 | 6 | 6 | | 34.8 | |
| | Delta | 1.2 | 1.8 | 0.9 | 1.2 | 2 | 1.2 | | | 8.3 |
| 2 | Pre-tx | 4.8 | 4.2 | 3.6 | 4.4 | 3.6 | 6 | 26.6 | | |
| | Post-tx | 5.4 | 5.4 | 5.7 | 5.6 | 4.8 | 6 | | 32.9 | |
| | Delta | 0.6 | 1.2 | 2.1 | 1.2 | 1.2 | 0 | | | 6.3 |
| 3 | Pre-tx | 4.8 | 4.2 | 3 | 2.4 | 6 | 6 | 26.4 | | |
| | Post-tx | 6 | 6 | 6 | 5.6 | 6 | 6 | | 35.6 | |
| | Delta | 1.2 | 1.8 | 3 | 3.2 | 0 | 0 | | | 9.2 |
| 4 | Pre-tx | 3.6 | 3.9 | 6 | 5.2 | 4 | 6 | 28.7 | | |
| | Post-tx | 6 | 6 | 6 | 6 | 6 | 6 | | 36 | |
| | Delta | 2.4 | 2.1 | 0 | 0.8 | 2 | 0 | | | 7.3 |
| 5 | Pre-tx | 2.4 | 3 | 4.8 | 2.4 | 2.4 | 6 | 21.0 | | |
| | Post-tx | 6 | 6 | 6 | 5.6 | 5.2 | 6 | | 34.8 | |
| | Delta | 3.6 | 3 | 1.2 | 3.2 | 2.8 | 0 | | | 13.8 |
| 6 | Pre-tx | 4.8 | 5.4 | 3.9 | 4.8 | 3.6 | 6 | 28.5 | | |
| | Post-tx | 5.4 | 6 | 6 | 4.8 | 4.8 | 5.2 | | 32.2 | |
| | Delta | 0.6 | 0.6 | 2.1 | 0 | 1.2 | 0.8 | | | 3.7 |
| 7 | Pre-tx | 4.8 | 3.9 | 3.9 | 4.8 | 4.8 | 4.8 | 27.0 | | |
| | Post-tx | 6 | 6 | 6 | 6 | 6 | 5.2 | | 35.2 | |
| | Delta | 1.2 | 2.1 | 2.1 | 1.2 | 1.2 | 0.4 | | | 8.2 |
| 8* | Pre-tx | 2.4 | 3 | 1.2 | 1.6 | 3.6 | 3.6 | 15.4 | | |
| | Post-tx | 6 | 6 | 5.1 | 5.2 | 6 | 5.6 | | 33.9 | |
| | Delta | 3.6 | 3 | 3.9 | 3.6 | 2.4 | 2 | | | 18.5 |
| 9* | Pre-tx | 5.4 | 3.9 | 1.5 | 1.2 | 4.8 | 4.4 | 21.2 | | |
| | Post-tx | 5.4 | 5.4 | 5.7 | 4 | 5.6 | 6 | | 32.1 | |
| | Delta | 0 | 1.5 | 4.2 | 2.8 | 0.8 | 1.6 | | | 10.9 |
| 10* | Pre-tx | 0 | 0 | 0 | 0.8 | 0 | 0 | 0.8 | | |
| | Post-tx | 1.2 | 0 | 0 | 0 | 0.8 | 0 | | 2 | |
| | Delta | 1.2 | 0 | 0 | −0.8 | 0.8 | 0 | | | 1.2 |
| 11* | Pre-tx | 0 | 0 | 0 | 0 | 0.8 | 0 | 0.8 | | |
| | Post-tx | 3.6 | 3.6 | 5.4 | 5.2 | 2.4 | 0 | | 20.2 | |
| | Delta | 3.6 | 3.6 | 5.4 | 5.2 | 1.6 | 0 | | | 19.4 |
| 12* | Pre-tx | 5.4 | 3 | 1.2 | 4.4 | 4 | 1.2 | 19.2 | | |
| | Post-tx | 4.8 | 5.7 | 6 | 6 | 6 | 6 | | 34.5 | |
| | Delta | 0.6 | 2.7 | 4.8 | 1.6 | 2 | 4.8 | | | 15.3 |
| 13* | Pre-tx | 4.2 | 4.5 | 3 | 2.4 | 3.2 | 5.2 | 22.5 | | |
| | Post-tx | 4.8 | 6 | 5.4 | 6 | 5.2 | 5.6 | | 33 | |
| | Delta | 0.6 | 1.5 | 2.4 | 3.6 | 2.0 | 0.4 | | | 10.5 |

(As compared to the corresponding table in the priority applications, some clerical errors in Table 1 have been corrected.)

Ten of the women were postmenopausal. Nine of the postmenopausal patients had been on hormone replacement therapy for at least six months before this treatment. One postmenopausal patient (#10) had not received hormone replacement. With an FSFI score below 26.55 pre-treatment, nine of the thirteen women satisfied the diagnostic criteria for Female Sexual Dysfunction before treatment.

Because the pre-injection and post-injection observations were for the same individuals, a paired sample t-test was used, assuming that differences between the paired observations were normally distributed.

All but one of the thirteen women showed significant improvement following treatment. One patient (#10) showed a small improvement in the overall score, but the change was not statistically significant in that instance. It may be significant that patient #10 suffered dyspareunia and was the only postmenopausal patient in the study who had not received hormone replacement therapy. Future studies will determine whether the novel therapy achieves better results in patients who have normal hormone levels or who are receiving hormone replacement therapy, or perhaps the outlier in this pilot study was just that, a statistical outlier. Put differently, had the inclusion criterion in the pilot study required patients to have normal hormone levels or hormone replacement/correction, then the results reported here would have been even better.

The mean of the individual domain scores improved for all 13 women across all domains, and those changes were statistically significant (p<0.05), except that the score for the Pain domain improved numerically but not significantly (p>0.05).

The mean overall pre-treatment score for all 13 women was 20.35. The mean score for all women increased to 30.56 after treatment, with an average improvement across all participants of 10.2 (p<0.05). A Wilcoxon signed-rank test, comparing overall scores before and after treatment, gave a W-statistic of 0.0 and a P-value of 0.002.

Of the 12 with significant improvement in overall scores, 9 (75%) had changed from a diagnosis of “female sexual dysfunction” to a diagnosis of “no dysfunction” (FSFI>26.55).

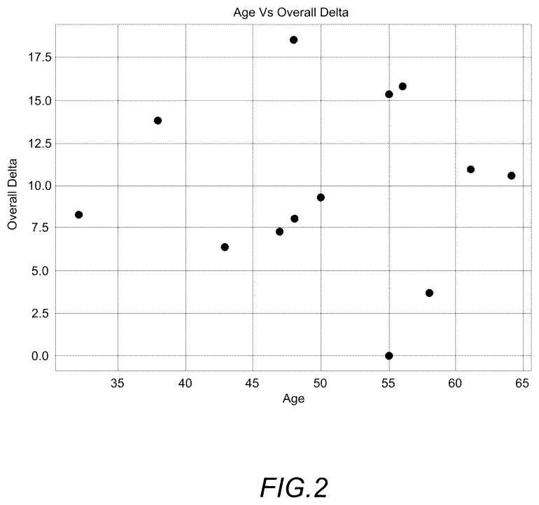

As shown in FIG. 2, the change in FSFI following BoNT injection appeared to be largely independent of the patient's age. Comparable responses were seen across all participating ages.

In summary, the results from the preliminary trial suggested that BoNT injections into the clitoris improve a woman's FSFI score by around 10 points, representing a significant improvement in sexual function.

### Effects of Adding PRP to BoNT

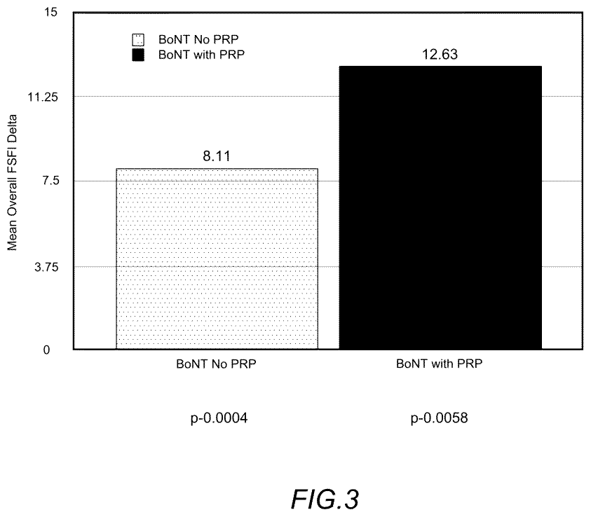

Six women chose to receive BoNT without PRP, and seven chose to receive BoNT with PRP added. A two-tailed, paired-sample t-test was used to determine whether there was a difference in the effects of treatment between these two groups. As seen in FIG. 3, both the PRP and Saline (non-PRP) groups showed statistically significant improvements in FSFI following injection with BoNT. Further, the group receiving both BoNT and PRP showed a statistically significant improvement over the group receiving BoNT in saline without PRP.

For the women receiving BoNT without PRP, the mean increase in overall FSFI was 8.11 (p=0.0004). For the women who received BoNT with PRP, the overall mean increase in FSFI was 12.63 (p=0.0058). The differences between the two groups, indicated by a t-statistic of 3.889 and a p-value of 0.006, suggest a statistically significant improvement in FSFI scores for the women treated with both BoNT and PRP as compared to those treated with BoNT but no PRP.

The overall improvement in FSFI for all 13 women, a Shapiro-Wilks test to compare pre- and post-treatment FSFI scores across all six domains, and the overall before-and-after scores all confirmed a normal distribution of the data and the validity of a paired sample t-test analysis.

The Box-Cox Method was also applied, and it also supported the conclusion that the data were distributed normally.

FDA approval for a new drug (or a new indication) for treating female sexual dysfunction is based in large part on whether improvement is seen in the Satisfaction domain. Correlation analyses were performed between the Satisfaction domain and each of the other domains. The following correlation coefficients were found: Desire (0.47), Arousal (0.56), Lubrication (−0.06), Orgasm (0.23), Pain (0.28). Thus, following treatment, an overall increase in Satisfaction correlated most strongly with improvements in Desire and Arousal, an intermediate correlation with Orgasm and Pain, but a low correlation with Lubrication.

We assessed the accuracy of the magnitude of the effect of treatment by calculating Cohen's d-value for the group treated with BoNT plus PRP and for the group treated with BoNT alone. The results were 2.31 for the BoNT-plus-PRP group, and 1.58 for the BoNT-only group. Both Cohen's d-values were greater than 0.8, suggesting a large treatment effect in both groups, with an even stronger effect in the combined PRP-plus-BoNT group. In comparing the two groups, Cohen's d-value was 0.856, also indicating a robust treatment enhancement by the addition of PRP (with a d-value greater than 0.8 indicating a large effect).

Future studies will further confirm the pilot-study results by comparing results for five treatment groups: (1) a shallow needle puncture of the clitoris, (2) a saline injection, (3) BoNT reconstituted with saline, (4) BoNT reconstituted with PRP, and (5) injection with PRP alone.

### Side Effects

Two women voluntarily reported, one week following the procedure, an overall subjective decrease in sexual function. However, four weeks post-procedure, both women instead reported subjectively improved function, and their FSFI demonstrated improvements over baseline. The temporary decrease in sexual function seen in two of the thirteen women is not reflected in the numbers reported above because the study did not score FSFI earlier than four weeks post-procedure.

By contrast, four of the women experienced hypersexuality for about a week immediately following injection, with arousal and the urge for orgasmic relief sometimes interrupting daily activities.

Eight of the women reported increased engorgement of the labia; one woman canceled a scheduled surgical procedure planned to augment the labia majora because she perceived that the clitoral injection of BoNT had triggered sufficient additional blood flow to the labia to achieve the desired enhancement.

Five of the thirteen women voluntarily reported a side effect of a subjective improvement in urge incontinence.

### Discussion

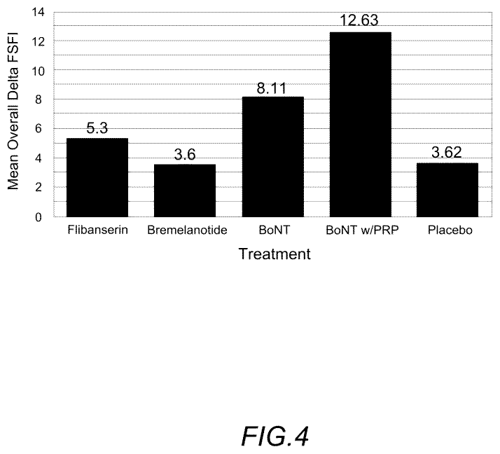

FIG. 4 compares the overall FSFI scores reported here with those from a metanalysis of other reported treatments (J. Weinberger et al., Female Sexual Dysfunction and the Placebo Effect: A Meta-analysis. Obstet. Gynecol. 2018; 132 (2): 453-458), namely, flibanserin or bremelanotide (the only two non-hormonal, FDA-approved treatments for female sexual dysfunction), and placebo. Both clitoral injection of BoNT and the injection of BoNT combined with PRP showed greater benefit than either of the two currently FDA-approved drugs.

Not only did desire, orgasm, arousal, lubrication, and overall sexual function improve in the present study, but satisfaction also improved, which is the approval-determining factor for the FDA. Therefore, the novel procedure offers hope for a possible new indication for BoNT for treating sexual dysfunction in women.

### Safety

There is a long-established safety profile for BoNT, with millions of doses administered each year for the past two decades (for other purposes/indications), in women of reproductive age, with few serious sequelae reported at the dosages that are used for in the current treatment. Indeed, BoNT is considered safe for use for cosmetic purposes at doses higher than those used in the present study. In addition to those warnings that are associated with the treatment of sexual dysfunction and injections in general, information provided for informed consent should also include the warnings that are typically used when BoNT is administered for aesthetic purposes, including warnings that it should not be used in the presence of pregnancy, myasthenia gravis, or known allergies to BoNT.

### Duration

Anecdotally, six women in the study reported an attenuation of effects and requested retreatment about six months after the first treatment. A higher dosage (e.g., 100 units) may afford longer-acting results. However, even should women need retreatment as frequently as every 12 weeks, that dosing schedule would not be more frequent than what is routinely done with BoNT for aesthetic and migraine indications. Should regenerative effects (neovascularization and neurogenesis) occur, then some aspects may be longer lasting and may be cumulative, perhaps delaying the future redevelopment of sexual dysfunction.

### Discussion

BoNT injected directly into the clitoris with the novel method improved sexual function in the domains of orgasm, arousal, and satisfaction. BoNT is an effective treatment for female sexual dysfunction. BoNT can be used alone, or in combination with PRP for synergy.

### Use of Clostridial Toxins—General Discussion

The following general discussion concerning clostridial toxins is adapted from U.S. patent publication US20120251518, and is incorporated herein to the extent not inconsistent with the foregoing, more specific disclosure describing the present inventor's discoveries and data. The preceding, more specific disclosure takes precedence over the following, more general disclosure to the extent that any conflicts may exist:

The ability of Clostridial toxins, such as, e.g., Botulinum neurotoxins (BoNTs), BoNT/A, BoNT/B, BoNT/C1, BoNT/D, BoNT/E, BoNT/F and BoNT/G, to inhibit neuronal transmission are being exploited in a wide variety of therapeutic and cosmetic applications. Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). As an example, BOTOX® has been approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasolabial lines, spasmodic dysphonia, strabismus, and VII nerve disorder.

Strains of Clostridium botulinum produce seven antigenically-distinct types of Botulinum toxins (BoNTs), which have been identified by investigating botulism outbreaks in man (BoNT/A, BoNT/B, BoNT/E and BoNT/F), animals (BoNT/C1 and BoNT/D), or isolated from soil (BoNT/G). BoNTs possess approximately 35% amino acid identity with each other and share the same functional domain organization and overall structural architecture. It is recognized by those of skill in the art that within each type of Clostridial toxin, subtypes can differ somewhat in their amino acid sequences and also in the nucleic acids encoding these proteins. For example, there are at least five known BoNT/A subtypes, BoNT/A1, BoNT/A2, BoNT/A3 BoNT/A4 and BoNT/A5, with specific subtypes showing approximately 89% amino acid identity as compared to another BoNT/A subtype. While all seven BoNT serotypes have similar structure and pharmacological properties, each also displays heterogeneous characteristics.

Clostridial toxins are released by Clostridial bacteria as complexes comprising the approximately 150-kDa Clostridial toxin along with associated non-toxin proteins (NAPs). Identified NAPs include proteins possessing hemagglutination activity, such, e.g., a hemagglutinin of approximately 17-kDa (HA-17), a hemagglutinin of approximately 33-kDa (HA-33) and a hemagglutinin of approximately 70-kDa (HA-70); as well as non-toxic non-hemagglutinin (NTNH), a protein of approximately 130-kDa. Thus, the botulinum toxin type A complex can be produced by Clostridial bacterium as 900-kDa, 500-kDa and 300-kDa forms. Botulinum toxin types B and C1 are apparently produced as only a 500-kDa complex. Botulinum toxin type D is produced as both 300-kDa and 500-kDa complexes. Finally, botulinum toxin types E and F are produced only as approximately 300-kDa complexes. The differences in molecular weight for the complexes are due to differing ratios of NAPs. The toxin complex is important for the intoxication process because it protects against adverse environmental conditions and is resistant to protease digestion. It also appears to facilitate internalization and activation of the toxin.

A Clostridial toxin is translated as a single-chain polypeptide that is subsequently cleaved by proteolytic scission within a disulfide loop by a naturally-occurring protease. This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond, as well as noncovalent interactions between the two chains. In some serotypes, such as, e.g., BoNT/A, the naturally-occurring protease is produced endogenously by the bacteria serotype, and cleavage occurs within the cell before the toxin is released into the environment. However, in other serotypes, such as, e.g., BoNT/E, the bacterial strain appears not to produce an endogenous protease capable of converting the single-chain form of the toxin into the di-chain form. In these situations, the toxin is released from the cell as a single-chain toxin, which is subsequently converted into the di-chain form by a naturally-occurring protease found in the environment.

Each mature di-chain molecule of a Clostridial toxin comprises three functionally distinct domains: 1) an enzymatic domain located in the light chain (LC) that includes a metalloprotease region with a zinc-dependent endopeptidase activity that specifically targets core components of the neurotransmitter release apparatus; 2) a translocation domain contained within the amino-terminal half of the heavy chain (HN) that facilitates release of the LC from intracellular vesicles into the cytoplasm of the target cell; and 3) a binding domain found within the carboxyl-terminal half of the heavy chain (HC) that determines the binding activity and binding specificity of the toxin to the receptor complex located at the surface of the target cell. The HC domain comprises two distinct structural features of roughly equal size that indicate function and are designated the HCN and HCC subdomains.

Clostridial toxins act on the nervous system by blocking the release of acetylcholine (ACh) at the pre-synaptic neuromuscular junction. These three functional domains, binding, translocation, and enzymatic activity, are all necessary for toxicity. While all details of this process are not yet precisely known, the overall cellular intoxication mechanism whereby Clostridial toxins enter a neuron and inhibit neurotransmitter release is similar, regardless of serotype or subtype. Although applicants have no wish to be limited by the following description, the intoxication mechanism has been described as comprising at least four steps: 1) receptor binding, 2) complex internalization, 3) light chain translocation, and 4) enzymatic target modification. The process is initiated when the binding domain of a Clostridial toxin binds to a toxin-specific receptor system located on the plasma membrane surface of a target cell. The binding specificity of a receptor complex is thought to be due in part to specific combinations of gangliosides and protein receptors that appear to distinctly comprise each Clostridial toxin receptor complex. Once bound, the toxin/receptor complexes are internalized by endocytosis, and the internalized vesicles are sorted to specific intracellular routes. The translocation step appears to be triggered by acidification of the vesicle compartment. This process seems to initiate pH-dependent structural rearrangements that increase hydrophobicity, creating a pore in the vesicle membrane and promoting the formation of the di-chain form of the toxin. Once di-chain formation occurs, light-chain endopeptidase of the toxin is released from the intracellular vesicle via the pore into the cytosol, where it appears to specifically target one of three known core components of the neurotransmitter release apparatus. These core proteins, vesicle-associated membrane protein (VAMP)/synaptobrevin, synaptosomal-associated protein of 25 kDa (SNAP-25), and syntaxin, are necessary for synaptic vesicle docking and fusion at the nerve terminal. They constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl terminus. The botulinum serotypes BoNT/B, BoNT/D, BoNT/F, and BoNT/G act on the conserved central portion of VAMP and release the amino-terminal portion of VAMP into the cytosol. BoNT/C1 cleaves Syntaxin at a single site near the cytosolic membrane surface.

Aspects of the present specification disclose, in part, a Clostridial toxin. As used herein, the term “Clostridial toxin” refers to any toxin produced by a Clostridial toxin strain that can execute the overall cellular mechanism whereby a Clostridial toxin intoxicates a cell and encompasses the binding of a Clostridial toxin to a low- or high-affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin such as BoNT/A, a BoNT/B, a BoNT/C1, a BoNT/D, a BoNT/E, a BoNT/F, or a BoNT/G. The BoNT/C2 cytotoxin and BoNT/C3 cytotoxins, not being neurotoxins, are excluded from the term “Clostridial toxin” as used here. A Clostridial toxin includes, without limitation, naturally occurring Clostridial toxin variants, such as, e.g., Clostridial toxin isoforms and Clostridial toxin subtypes; non-naturally occurring Clostridial toxin variants, such as e.g., conservative Clostridial toxin variants, non-conservative Clostridial toxin variants, Clostridial toxin chimeric variants and active Clostridial toxin fragments thereof, or any combination thereof.

A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C1 complex, a 500-kDa BoNT/D complex, a 300-kDa BoNT/D complex, a 300-kDa BoNT/E complex, and a 300-kDa BoNT/F complex.

Clostridial toxins are available commercially. Alternatively, they can be produced using standard purification or recombinant biology techniques known to those skilled in the art. See, e.g., Hui Xiang et al., Animal Product Free System and Process for Purifying a Botulinum Toxin, U.S. Pat. No. 7,354,740, which is hereby incorporated by reference in its entirety. For example, a BoNT/A complex can be isolated and purified from anaerobic fermentation by cultivating Clostridium botulinum type A in a suitable medium. Raw toxin can be harvested by precipitation with sulfuric acid and concentrated by ultramicrofiltration. Purification can be carried out by dissolving the acid precipitate in calcium chloride. The toxin can then be precipitated with cold ethanol. The precipitate can be dissolved in sodium phosphate buffer and centrifuged. After drying, approximately 900 kD crystalline BoNT/A complex can be obtained with a specific potency of 3×10⁷ LD₅₀ U/mg or greater. Furthermore, NAPs can be separated to obtain purified toxin, such as e.g., BoNT/A with an approximately 150 kD molecular weight with a specific potency of 1-2×10⁸ LD₅₀ U/mg or greater, purified BoNT/B with an approximately 156 kD molecular weight with a specific potency of 1-2×10⁸ LD₅₀ U/mg or greater, and purified BoNT/F with an approximately 155 kD molecular weight with a specific potency of 1-2×10⁷ LD₅₀ U/mg or greater. See Edward J. Schantz & Eric A. Johnson, Properties and use of Botulinum Toxin and Other Microbial Neurotoxins in Medicine, Microbiol. Rev. 56:80-99 (1992). As another example, recombinant Clostridial toxins can be recombinantly produced as described in U.S. Patent Publication 2008/0057575 or 2008/0138893.

Clostridial toxins available commercially include BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). Clostridial toxin complexes may be obtained from, e.g., List Biological Laboratories, Inc. (Campbell, Calif.), the Centre for Applied Microbiology and Research (Porton Down, U.K), Wako (Osaka, Japan), and Sigma Chemicals (St Louis, Mo.).

A composition disclosed herein is generally administered as a pharmaceutical acceptable composition. As used herein, the term “pharmaceutically acceptable” means any molecular entity or composition that does not produce an adverse, allergic, or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the Clostridial toxins rein. A pharmaceutical composition may be administered alone, or in combination with other supplementary active ingredients, agents, drugs, or hormones. The pharmaceutical composition may be manufactured in any dosage form suitable for administration.

A pharmaceutical composition disclosed herein may optionally include a pharmaceutically acceptable carrier that facilitates the processing of an active ingredient into pharmaceutically acceptable compositions. As used herein, the term “pharmacologically acceptable carrier” is synonymous with “pharmacological carrier” and means any carrier that has substantially no long-term or permanent detrimental effect when administered and encompasses terms such as “pharmacologically acceptable vehicle, stabilizer, diluent, additive, auxiliary or excipient.” Such a carrier generally is mixed with an active ingredient, or permitted to dilute or enclose the active compound and can be a solid, semi-solid, or liquid agent. It is understood that the active ingredients can be soluble or can be delivered as a solution or suspension in the desired carrier or diluent. Any of a variety of pharmaceutically acceptable carriers can be used, including, without limitation, aqueous media such as, e.g., water, saline, glycine, hyaluronic acid, and the like; solvents; dispersion media; coatings; antibacterial and antifungal agents; isotonic and absorption delaying agents; or any other inactive ingredient. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); GOODMAN & GILMAN'S THE PHARMACOLOGICAL BASIS OF THERAPEUTICS (Joel G. Hardman et al., eds., McGraw-Hill Professional, 10th ed. 2001); and HANDBOOK OF PHARMACEUTICAL EXCIPIENTS (Raymond C. Rowe et al., APhA Publications, 4th edition 2003). These protocols are routine procedures, and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, emulsifying agents, wetting agents, anticoagulants, autologous plasma or other autologous components, and the like. Various buffers and means for adjusting pH can be used to prepare a pharmaceutical composition, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelating agents, such as, e.g., DTPA or DTPA-bisamide, calcium DTPA, and CaNaDTPA-bisamide. Pharmaceutically acceptable anticoagulants include, without limitation, sodium citrate or acid citrate dextrose solution (ACD-A or B). Autologous substances that are useful include, without limitation, platelet-rich plasma (PRP), whole blood, micronized fat, bone marrow-derived mesenchymal cells, and stem cells. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt which can be formed with many mineral or organic acids, including but not limited to hydrochloric, sulfuric, acetic, lactic, tartaric, malic, succinic acids, etc. Such salts often tend to be more soluble in aqueous solvent than are the corresponding free base forms. It is understood that these and other substances known in the art of pharmacology can be included in a pharmaceutical composition.

In another embodiment, a composition is a pharmaceutical composition comprising a Clostridial toxin. In aspects of this embodiment, a pharmaceutical composition comprising a Clostridial toxin further comprises a pharmacological carrier, a pharmaceutical component, or both a pharmacological carrier and a pharmaceutical component. In other aspects of this embodiment, a pharmaceutical composition comprising a Clostridial toxin further comprises at least one pharmacological carrier, at least one pharmaceutical component, or at least one pharmacological carrier and at least one pharmaceutical component.

Aspects of the present specification disclose, in part, treating an individual suffering from a sexual dysfunction disorder. As used herein, the term “treating” refers to reducing or eliminating in an individual a clinical symptom of a sexual dysfunction disorder or delaying or preventing in an individual the onset of a clinical symptom of a sexual dysfunction disorder. For example, the term “treating” can mean reducing a symptom of a condition characterized by a sexual dysfunction disorder by, e.g., at least 20%, at least 30%, at least 40%, at least 50%, at least 60%, at least 70%, at least 80%, at least 90% or at least 100%. The actual symptoms associated with a sexual dysfunction disorder are well known and can be determined by a person of ordinary skill in the art by taking into account factors, including, without limitation, the location of the sexual dysfunction disorder, the cause of the sexual dysfunction disorder, the severity of the sexual dysfunction disorder, and/or the tissue or organ affected by the sexual dysfunction disorder. Those of skill in the art will understand the appropriate symptoms or indicators associated with specific sexual dysfunction disorders and will understand how to determine when an individual is a candidate for treatment as disclosed herein.

As used herein, the term “sexual dysfunction” or “sexual dysfunction disorder” refers to a situation in which at least one of the underlying symptoms being treated is due to one or more of a sensory nerve-based etiology, a sympathetic nerve-based etiology, or a parasympathetic nerve-based etiology. Typically, such etiologies will involve an abnormal overactivity or underactivity of a nerve that results in symptoms of a sexual dysfunction disorder or any normal activity of a nerve that needs to be reduced or stopped for a period of time, or increased for a period of time, in order to treat a sexual dysfunction disorder. Such etiologies may also involve an imbalance of the autonomic nervous system with sympathetic overactivity or underactivity and parasympathetic overactivity or underactivity. Other factors affecting sex include endocrine function, cardiac function, side effects of medications, and psychiatric and social factors. In short, almost anything that affects health can also affect sexual function. A sexual dysfunction disorder can be a condition that makes difficult, reduces, or prevents an individual's enjoyment of normal sexual activity, including, e.g., desire, arousal, lubrication, or orgasm; or makes difficult, reduces, or prevents the normal physiological changes brought on normally by such activity. Sexual dysfunction disorders include, without limitation, a sexual desire disorder, a sexual arousal disorder, a sexual orgasm disorder, a sexual pain disorder, a sexual lubrication disorder, a sexsomnia, and a climacturia.

A sexual desire disorder refers to a sexual dysfunction disorder where an individual has an abnormal desire or libido for sexual thoughts or fantasies and/or desire for sexual activity and includes both a complete aversion to sexual thoughts, fantasies, or activities, and a complete preoccupation with sexual thoughts, fantasies, or activities. A sexual desire disorder includes situations where there is a significant difference in desire or libido between an individual and their partner or where an individual alone has an abnormal distaste for or fixation with sexual thoughts, fantasies, or activities. Sexual desire disorders include, without limitation, a hypoactive sexual desire disorder, a sexual aversion disorder, and a hyperactive sexual desire disorder.

A hypoactive sexual desire disorder (also known as inhibited sexual desire disorder) refers to a condition where sexual thoughts or fantasies and/or desire for sexual activity is persistently reduced or absent. The condition ranges from a reduced or complete lack of sexual desire to engage in any type of sexual thought, fantasy, or activity with the current partner to a reduced or complete lack of sexual desire to engage in any type of sexual thought, fantasy, or activity generally. The condition may have started after a period of normal sexual functioning, or the individual may always have had no/low sexual desire. An individual with a hypoactive sexual desire disorder has little or no interest in engaging in any type of sexual activity, has few sexual thoughts or fantasies, and generally has a lack of sexual response.

A sexual aversion disorder refers to a condition where an individual is unusually apprehensive or repulsed by the thought of engaging in any type of sexual thoughts, fantasies, or activity. The condition ranges from an unusual apprehension or repulsion to engage in any type of sexual thought, fantasy, or activity with the current partner to having such apprehension or repulsion generally. The condition may have started after a period of normal sexual functioning, or the individual may always have had an aversion to sexual thoughts, fantasies, and/or activities. An individual with a sexual aversion disorder has little or no interest in engaging in any type of sexual activity, avoids genital sexual contact, has few or no sexual thoughts or fantasies, and has a general lack of sexual response.

A hyperactive sexual desire disorder (also known as hypersexual desire disorder, sex addiction, sexual compulsivity, nymphomania, or satyriasis) refers to a condition where sexual thoughts or fantasies and/or desire for sexual activity are abnormally persistent in an individual. This disorder lies on the far end of the spectrum from hypoactive sexual desire disorder. An individual with a hyperactive sexual desire disorder typically experiences significant personal distress or impairment in social, occupational, or other important areas of functioning. The condition ranges from having sexual thoughts or fantasies and/or the desire to engage in any type of sexual activity with the current partner to having such thoughts, fantasies and/or desires generally. The condition may have started after a period of normal sexual functioning or the individual may always have had a persistent or heightened sexual desire. An individual with a hyperactive sexual desire disorder is constantly interested in engaging in any sexual activity; is preoccupied with sexual thoughts or fantasies; has recurrent and intense sexual thoughts, urges or fantasies; repeatedly has sexual thoughts, fantasies, or urges in response to anxiety, depression, boredom, irritability, or stress; is preoccupied with planning or engaging in a sexual activity; is very demanding sexually; has a quick sexual response; abnormally engages in sexual activity; repeatedly engages in sexual activity in response to anxiety, depression, boredom, irritability, or stress; repeatedly being unsuccessful at trying to control sexual thoughts, fantasies, urges, or behavior; and repeatedly engaging in sexual activity despite physical or emotional risk to individual or others.

A sexual arousal disorder refers to a sexual dysfunction disorder where an individual has an abnormal arousal response to sexual stimulation, including, but not limited to, physical, emotional, and/or mental stimulation. A sexual arousal disorder includes situations where there is an abnormal arousal response to sexual stimulation from a partner or from the individual. Sexual arousal disorders include, without limitation, a deficient sexual arousal disorder, a persistent sexual arousal disorder, and priapism.

A deficient sexual arousal disorder refers to a condition where there is a lack of an arousal response in an individual after sexual stimulation. These disorders include situations where physical sexual stimulation fails to evoke a sexual response in an individual or where, even though the individual has a desire for sexual activity and is physically aroused, the individual does not feel sexually aroused.

A deficient sexual arousal disorder can be classified into several types, which can occur alone or in combination with other disorders. Subjective deficient sexual arousal disorders refer to conditions where, regardless of the amount or degree of sexual stimulation, the individual feels no or little mental sexual arousal. Thus, a woman may exhibit increased vaginal lubrication but still does not feel sexually aroused. Genital deficient sexual arousal disorders refer to conditions where, regardless of the amount or degree of sexual stimulation, the individual physically exhibits no or minimal arousal response even though the individual has the desire to engage in sexual activity. Thus, a woman can exhibit no vaginal lubrication or swelling of the vulva. Combined genital and subjective deficient arousal disorders refer to conditions where regardless of the amount or degree of sexual stimulation, there is no or minimal physical manifestation of an arousal response, and the individual has no or minimal mental sexual arousal.

Frigidity is considered a sexual arousal disorder, and may be further characterized by terms describing specific issues. This disorder involves the persistent or recurrent inability to reach or sustain the lubrication and swelling reaction in the arousal phase of the sexual response to the point that it causes personal distress. It is the second most common sexual problem among women, affecting an estimated 20% of women, and most frequently occurs in postmenopausal women, but can occur at any age.

Low estrogen or low testosterone levels after menopause can make vaginal tissue dry and thin and reduce blood flow to the genitals. As a result, the arousal phase of the sexual response may take longer and the sensitivity of the vaginal area may decline.

Persistent sexual arousal disorder (also known as persistent genital arousal disorder, persistent sexual arousal syndrome, restless genital syndrome, and mempin syndrome) refers to a condition where there is spontaneous, persistent, and uncontrollable genital arousal in the absence of any sexual stimulation. This arousal is unrelated to any sexual thoughts or desires, can occur with or without genital engorgement and/or orgasm, and can last for hours or even days. An individual with persistent sexual arousal disorder experiences sexual arousal lasting for an extended period of time; the arousal typically does not diminish on its own; the sexual arousal is not related to sexual desire or stimulation; the sexual arousal can be triggered by nonsexual events or by nothing at all; there can be persistent physical signs of sexual arousal after orgasm; or there can be dissipation of physical signs of sexual arousal only after multiple orgasms, and there can be intrusive and unwanted sexual arousal, or distress.

A sexual orgasm disorder refers to a sexual dysfunction disorder where an individual experiences an abnormal orgasm. Sexual orgasmic disorders generally fall into one of several categories, including those where, during normal sexual activity, orgasms are absent (anorgasmia), orgasms are delayed or difficult to achieve, orgasms are too rapid, or orgasms have diminished sensation.

In females, common sexual orgasm disorders include female orgasmic disorder, female anorgasmia, and inhibited female orgasm. A female orgasmic disorder is a condition where there is a delay in orgasm even following sufficient sexual stimulation and arousal. The disorder can have physical, psychological, or pharmacological origins.

A female anorgasmia disorder refers to a condition where there is an absence of orgasm even after sufficient sexual stimulation and arousal. Female anorgasmia is further broken down into primary anorgasmia, which refers to a woman having never had an orgasm; and secondary anorgasmia, where a woman has previously experienced orgasms but no longer does, or where she can only experience orgasms under specific conditions, such as e.g., only through masturbation but not during sexual intercourse. A female anorgasmia disorder may be temporary or persistent. The disorder can have physical, psychological, or pharmacological origins.

Inhibited female orgasm refers to a condition where there is a delay or absence in reaching orgasm after sufficient sexual stimulation and arousal. The disorder can have physical, psychological, or pharmacological origins.

A sexual pain disorder refers to a sexual dysfunction disorder where pain typically occurs during or after sex, and may happen in response to arousal, to stimulation, or to orgasm. Both men and women report sexual pain disorders, but they are reported much more often in women. Sexual pain disorders include, without limitation, dyspareunia, vaginismus, vulvodynia, and dysorgasmia.

Dyspareunia refers to a condition where pain is experienced during sexual intercourse. Dyspareunia can have physical and/or emotional causes. The most common cause of pain during sex is inadequate vaginal lubrication (vaginal dryness), occurring from a lack of arousal, medications, or hormonal changes. Painful sex also can be a sign of illness, infection, cysts, or tumors requiring medical treatment or surgery. Irritation from contraceptive creams and foams can also cause dryness, as can fear and anxiety about sex. The muscles of the pelvic floor can also be source of pain. An individual with dyspareunia experiences pain during sexual activity, has little or no interest in engaging in any type of sexual activity, and generally has a lack of sexual response.

Vaginismus refers to a condition where involuntary spasmodic muscle contractions occur at the entrance to the vagina, making it painful for anything to enter the vagina. When sexual intercourse is attempted despite these contractions, a painful experience results. Although the cause of vaginismus is currently unknown, both physical and psychological factors are suspected to play a role. An individual with vaginismus experiences pain during sexual activity, has little or no interest in engaging in any type of sexual activity, and generally has a lack of sexual response.

Vulvodynia (also known as vulvar vestibulitis) refers to a condition where pain is experienced in the vulva. It also includes pain outside the vulva on the labia or an itching, burning or sharp pain within the vulva. In this sexual pain disorder, a female will experience burning pain during sex which seems to be related to problems with the skin in the vulvar and vaginal areas. The cause of vulvodynia is currently unknown. Although the cause of vulvodynia is currently unknown, both physical and psychological factors are suspected to play a role. An individual with vulvodynia experiences pain during sexual activity, has little or no interest in engaging in any sexual activity, and generally has a lack of sexual response.

Dysorgasmia (also known as orgasmic pain) refers to a condition where pain is experienced during orgasm. An individual with dysorgasmia experiences pain during sexual activity, has little or no interest in engaging in any type of sexual activity, and generally has a lack of sexual response.

Sexsomnia (also known as sleep sex) refers to a sexual dysfunction disorder where sexual behaviors are initiated and occur while an individual is asleep. Such behaviors can occur while alone, in the presence of a partner, or with a partner. Sexsomnia is a type of sleep disorder (parasomnia) that involves sexual behaviors. Sexsomnia encompasses a range of sexual behaviors, including, but not limited to, sexual touching of another person, sexual vocalizations (moaning) and talking, masturbation (with and without orgasm), performing oral sex (with and without orgasm), and performing sexual intercourse (with and without orgasm). The current understanding is that the sexual behaviors are involuntary because the sleeping individual initiating them is unaware they are happening, and often has no memory of them occurring when confronted at a later point with the behavior.

Climacturia refers to a sexual dysfunction disorder where there is an involuntary release of urine at the time of orgasm.

Typically, any woman who is a candidate for a conventional sexual dysfunction disorder treatment is a candidate for a sexual dysfunction disorder treatment disclosed herein.

An effective amount of a Clostridial toxin is one for which the amount of the Clostridial toxin administered achieves the desired therapeutic effect. This amount may vary by individual, and can be determined for specific individuals by routine experimentation. For example, typically, about 50 U of BOTOX® (Allergan, Inc., Irvine, Calif.), or of Xeomin® (Merz North America, Inc., Raleigh, NC) a BoNT/A, is administered in order to treat a sexual dysfunction disorder, up to 75 U, 100 U, 150 U, 200 U, or even higher amounts.

A low-dose therapy may be sufficient for some individuals. For example, typically, about 75-150 U of BoNT is administered to treat a sexual dysfunction disorder. However, in a low-dose therapy, a smaller effective amount of BoNT could be used, for example, less than 50 U, less than 25 U, less than 15 U, less than 10 U, less than 7.5 U, less than 5 U, less than 2.5 U, or even less than 1 U.

The appropriate, effective amount of a Clostridial toxin to be administered to an individual for a particular sexual dysfunction disorder can be determined by a person of ordinary skill in the art by taking into account factors including, without limitation, the type of sexual dysfunction disorder, the location of the sexual dysfunction disorder, the cause of the sexual dysfunction disorder, the severity of the sexual dysfunction disorder, the degree of relief desired, the duration of relief desired, the particular Clostridial toxin used, the rate of excretion of the particular Clostridial toxin used, the pharmacodynamics of the particular Clostridial toxin used, the nature of the other compounds to be included in the composition, the particular route of administration, the particular characteristics, history and risk factors of the individual, such as, e.g., age, weight, general health and the like, or any combination thereof. Additionally, where repeated administration of a composition disclosed herein is used, an effective amount of a Clostridial toxin will further depend upon factors including, without limitation, the frequency of administration, the biological half-life of the particular Clostridial toxin, or any combination thereof.

Variations in what constitutes an effective amount are to be expected among patients, and individual therapeutically effective dosage levels and patterns are preferably determined by the attending physician in consideration of the above factors. The condition of an individual can be monitored throughout a course of therapy, and the effective amount for the individual can be adjusted accordingly.

In some embodiments of the invention, a therapeutically effective amount of a composition in accordance with the invention reduces a symptom associated with a sexual dysfunction disorder by, e.g., at least 10%, at least 20%, at least 30%, at least 40%, at least 50%, at least 60%, at least 70%, at least 80%, at least 90% or even 100%. In other embodiments, a therapeutically effective amount reduces a symptom associated with a sexual dysfunction disorder by, e.g., at most 10%, at most 20%, at most 30%, at most 40%, at most 50%, at most 60%, at most 70%, at most 80%, at most 90% or at most 100%. In yet other embodiments, a therapeutically effective amount reduces a symptom associated with a sexual dysfunction disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other embodiments, a therapeutically effective amount is a dosage sufficient to induce the desired outcome for, e.g., at least one week, at least one month, at least two months, at least three months, at least four months, at least five months, at least six months, at least seven months, at least eight months, at least nine months, at least ten months, at least eleven months, or at least twelve months.

In other embodiments, a therapeutically effective amount of a Clostridial toxin generally is in the range of about 1 fg to about 30.0 μg. In other aspects of this embodiment, a therapeutically effective amount of a Clostridial toxin can be, e.g., at least 1.0 pg, at least 10 pg, at least 100 pg, at least 1.0 ng, at least 10 ng, at least 100 ng, at least 1.0 μg, at least 10 μg, at least 100 μg, or at least 1.0 mg. In still other aspects, a therapeutically effective amount of a Clostridial toxin can be, e.g., at most 1.0 pg, at most 10 pg, at most 100 pg, at most 1.0 ng, at most 10 ng, at most 100 ng, at most 1.0 μg, at most 10 μg, at most 100 μg, or at most 1.0 mg. In still other aspects, a therapeutically effective amount of a Clostridial toxin can be, e.g., about 1.0 pg to about 10 μg, about 10 μg to about 10 μg, about 100 μg to about 10 μg, about 1.0 ng to about 10 μg, about 10 ng to about 10 μg, or about 100 ng to about 10 μg. In still other aspects, a therapeutically effective amount of a Clostridial toxin can be from, e.g., about 1.0 pg to about 1.0 μg, about 10 μg to about 1.0 μg, about 100 μg to about 1.0 μg, about 1.0 ng to about 1.0 μg, about 10 ng to about 1.0 μg, or about 100 ng to about 1.0 μg. In other aspects, a therapeutically effective amount of a Clostridial toxin can be from, e.g., about 1.0 pg to about 100 ng, about 10 μg to about 100 ng, about 100 μg to about 100 ng, about 1.0 ng to about 100 ng, or about 10 ng to about 100 ng.

In other aspects, a therapeutically effective amount of a Clostridial toxin generally is in the range of about 0.1 U to about 2500 U. In other aspects, a therapeutically effective amount of a Clostridial toxin can be, e.g., at least 1.0 U, at least 10 U, at least 50 U, at least 100 U, at least 250 U, at least 500 U, at least 750 U, at least 1,000 U, at least 1,500 U, at least 2,000 U, or at least 2,500 U. In still other aspects, a therapeutically effective amount of a Clostridial toxin can be, e.g., at most 1.0 U, at most 10 U, at most 50 U, at most 100 U, at most 250 U, at most 500 U, at most 750 U, at most 1,000 U, at most 1,500 U, at most 2,000 U, or at most 2,500 U. In still other aspects of this embodiment, a therapeutically effective amount can be, e.g., about 1 U to about 2,000 U, about 10 U to about 2,000 U, about 50 U to about 2,000 U, about 100 U to about 2,000 U, about 500 U to about 2,000 U, about 1,000 U to about 2,000 U, about 1 U to about 1,000 U, about 10 U to about 1,000 U, about 50 U to about 100 U, about 50 U to about 1,000 U, about 100 U to about 1,000 U, about 500 U to about 1,000 U, about 1 U to about 500 U, about 10 U to about 500 U, about 50 U to about 500 U, about 100 U to about 500 U, about 1 U to about 100 U, about 10 U to about 100 U, about 50 U to about 100 U, about 0.1 U to about 1 U, about 0.1 U to about 5 U, about 0.1 U to about 10 U, about 0.1 U to about 15 U, about 0.1 U to about 20 U, about 0.1 U to about 25 U.

In still other embodiments, a therapeutically effective amount of a Clostridial toxin generally is in the range of about 0.0001 U/kg to about 3,000 U/kg. In some aspects, a therapeutically effective amount of a Clostridial toxin can be, e.g., at least 0.001 U/kg, at least 0.01 U/kg, at least 0.1 U/kg, at least 1.0 U/kg, at least 10 U/kg, at least 100 U/kg, or at least 1000 U/kg. In other aspects, a therapeutically effective amount of a Clostridial toxin can be, e.g., at most 0.001 U/kg, at most 0.01 U/kg, at most 0.1 U/kg, at most 1.0 U/kg, at most 10 U/kg, at most 100 U/kg, or at most 1000 U/kg. In yet other aspects, a therapeutically effective amount of a Clostridial toxin can be between, e.g., about 0.001 U/kg to about 1 U/kg, about 0.01 U/kg to about 1 U/kg, about 0.1 U/kg to about 1 U/kg, about 0.001 U/kg to about 10 U/kg, about 0.01 U/kg to about 10 U/kg, about 0.1 U/kg to about 10 U/kg about 1 U/kg to about 10 U/kg, about 0.001 U/kg to about 100 U/kg, about 0.01 U/kg to about 100 U/kg, about 0.1 U/kg to about 100 U/kg, about 1 U/kg to about 100 U/kg, or about 10 U/kg to about 100 U/kg. As used herein, the term “unit” or “U” refers to the LD₅₀ dose, which is defined as the amount of a Clostridial toxin that kills 50% of the mice injected intraperitoneally with the Clostridial toxin.

Dosing can be single dosage or cumulative (serial dosing), and can be readily determined by one skilled in the art. For instance, treatment of a sexual dysfunction disorder may comprise a one-time administration of an effective dose of a composition disclosed herein. As a non-limiting example, an effective dose of a composition disclosed herein can be administered once to an individual, e.g., as a single injection. Alternatively, treatment of a sexual dysfunction disorder may comprise multiple administrations of an effective dose of a composition disclosed herein carried out over a range of time periods, such as, e.g., daily, once every few days, weekly, monthly, or yearly. As a non-limiting example, a composition disclosed herein can be administered once, twice, three times, four times, five times, or six times yearly to a woman. The timing of administration can vary from individual to individual, depending upon such factors as the severity of an individual's symptoms. For example, an effective dose of a composition disclosed herein can be administered to an individual once a month for an indefinite period of time, or until the individual no longer requires therapy. A person of ordinary skill in the art will recognize that the condition of the individual can be monitored throughout the course of treatment and that the effective amount of a composition disclosed herein that is administered can be adjusted accordingly.

The current preferred method for administering BoNT-containing preparations is by injection through a needle directly into the target tissues. Other methods known in the art for introducing drugs directly into target tissues may also be used, and are also considered to be “injections” within the scope of this invention. Besides the use of hypodermic needles, other methods of “injection” that can be used in the present invention include, for example, the following: Iontophoresis, using a small electric current to drive charged drug molecules through the skin or mucous membranes and into the underlying tissues; Jet injectors, using a high-pressure, air-driven mechanism to inject; Microneedle patches: tiny, painless needles on a patch that penetrate the skin's outer layer; Electroporation, using short electrical pulses to temporarily open pores in cell membranes, allowing drugs to enter the cells; Ultrasound-Assisted Delivery (Sonophoresis), Ultrasound waves create temporary disruptions in the skin or tissue, enhancing the absorption of drug; Laser-Assisted Drug Delivery: Lasers create microscopic channels in the skin to facilitate drug penetration; Needle-Free Liquid Jet Systems, Similar to jet injectors but using liquid pressure instead of gas to drive medications into the skin; Magnetophoresis, using magnetic fields to guide magnetic nanoparticles carrying drugs to specific tissues; Hydrogel-Based Delivery, hydrogels loaded with drugs are applied to the skin or inserted into the body, allowing controlled release over time; Electromagnetic Drug Delivery, Magnetic or electromagnetic fields are used to guide charged drug particles or enhance absorption; Transdermal Delivery (Other than Patches): Drug delivery through the skin using enhancers such as chemical penetration agents, thermal energy, or microdermabrasion; Electrospinning, drugs are encapsulated in nanofibers that dissolve upon contact with tissues; Implantable Devices, small devices or reservoirs implanted under the skin release medications over time; or Thermal Ablation, Heat energy temporarily disrupts skin barriers, allowing medications to enter tissues more deeply.

A composition disclosed herein can also be co-administered in combination with other therapeutic compounds, or with autologous blood products such as stem cells, adipocytes, platelet rich plasma (PRP).

Unless otherwise indicated or otherwise clearly implied by context, all numerical values associated with a characteristic, item, quantity, parameter, property, term, and so forth should be understood to be modified by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth are approximations that may vary. At the very least, and not as an attempt to limit the application of the doctrine of equivalents, each numerical indication should at least be construed in light of the number of reported significant digits and by applying ordinary rounding principles. Notwithstanding that the numerical ranges and values setting forth the broad scope of the invention are approximations, the numerical ranges and values set forth in the working examples are reported as precisely as possible. Any numerical range or value, however, inherently contains certain errors necessarily resulting from measurements and other sources of variation known in the art. Recitation of numerical ranges of values herein is intended to serve as a shorthand method of referring to all numerical values within the range. Unless otherwise indicated herein, each individual value within a numerical range is incorporated into the present specification as if it were individually recited herein.

The terms “a,” “an,” “the” and similar referents used in the context of describing the present invention are to be construed to cover both the singular and the plural, unless otherwise indicated herein or clearly contradicted by context. The steps for all methods described herein can be performed in any suitable order unless otherwise indicated herein or otherwise clearly contradicted by context. The use of examples, or exemplary language (e.g., “such as”) is intended merely to better illuminate the present invention and is not intended to limit the scope of the invention otherwise claimed. No language in the present specification should be construed as indicating that any non-claimed element should be considered essential to the practice of the invention.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. In the event of a conflict, the present disclosure takes precedence over an item incorporated by reference.

Also incorporated by reference are the complete disclosures of the priority applications: U.S. provisional application Ser. No. 63/551,599, filed 9 Feb. 2024; and U.S. provisional application Ser. No. 63/558,183, filed 27 Feb. 2024. In the event of a conflict, the present disclosure takes precedence over material incorporated by reference.

1. A method for treating a condition in a woman; wherein the condition comprises one or more dysfunctions selected from the group consisting of: hypoactive sexual desire, hypoactive sexual arousal, hypoactive clitoral tumescence, hypoactive sexual sensation, anorgasmia, inhibited orgasm, hypoactive sexual lubrication, and hypoactive sexual satisfaction; wherein said method comprises injecting directly into the woman's clitoris a therapeutically effective amount of a composition comprising a botulinum toxin; and wherein administration of the composition ameliorates one or more symptoms of the condition, thereby treating the woman.

2. The method of claim 1, wherein about 50 to about 250 units of the botulinum toxin are injected.

3. The method of claim 1, wherein about 50 units of botulinum toxin are injected.

4. The method of claim 1, wherein about 100 units of botulinum toxin are injected.

5. The method of claim 1, wherein the composition that is injected also comprises autologous, platelet-rich plasma.

6. The method of claim 1, wherein said injecting step is repeated at intervals of one to twelve months.

7. The method of claim 1, wherein said injecting step comprises injecting the body of the clitoris.

8. The method of claim 1, wherein said injecting step comprises injecting the corpus cavernosum.

9. A method comprising injecting a composition comprising a botulinum toxin directly into a woman's clitoris.

10. The method of claim 9, wherein 50 to 250 units of botulinum toxin are injected.

11. The method of claim 9, wherein about 50 units of botulinum toxin are injected.

12. The method of claim 9, wherein about 100 units of botulinum toxin are injected.

13. The method of claim 9, wherein the composition that is injected also comprises autologous, platelet-rich plasma.
